(19) United States
(12) Patent Application Publication
(10) Pub. No.: US 2026/0301160 A1
(43) Pub. Date: Oct. 1, 2026

(54) PRESSURE GENERATING DEVICE FOR BLASTOCYST FORMATION BOOSTING

(71) Applicant: Inti Taiwan, Inc., Zhubei City (TW)

(72) Inventors: Wei-Ming Chen, Zhubei City (TW); I-Chiao Hsieh, Zhubei City (TW); Chung-Li Chiang, Zhubei City (TW)

(21) Appl. No.: 19/091,144

(22) Filed: Mar. 26, 2025

(51) Int. Cl. G06T 7/00; C12M 1/42; C12N 5/075; G06V 10/77; G06V 10/82; G06V 20/69

(52) U.S. Cl. CPC G06T 7/0012; C12M 35/04; C12N 5/0609; G06V 10/7715; G06V 10/82; G06V 20/695; G06T 2207/10016; G06T 2207/10056; G06T 2207/30024

(57) ABSTRACT

The present disclosure generally relates to systems and methods for boosting blastocyst formation by applying pressure to oocytes. In some implementation examples, pressure is applied to an oocyte through a micropipette. An image sequence depicting the oocyte and the micropipette applying the pressure to the oocyte is obtained. Using a segmentation model, objects associated with the oocyte can be identified. Based on the objects identified, features such as morphological features and an aspiration depth associated with the oocyte can be determined. At least some of the features can then be fed into a machine learning model to generate an oocyte grade that indicates a likelihood of the oocyte developing into a usable blastocyst.

### FIELD

The disclosure relates to a pressure generating device, and more particularly to boosting blastocyst formation using a pressure generating device associated with an oocyte quality analysis system.

### BACKGROUND

In vitro fertilization (IVF) is an option for achieving pregnancy for couples suffering from infertility. In an IVF cycle, after stimulation of the ovaries of the mother using follicle stimulating hormone, several oocytes can be harvested from the ovaries and fertilized in vitro, and then one or more of the fertilized oocytes may be transferred back to the mother. In some cases, the fertilized oocytes may be further developed into embryos in vitro, and then one or more of the embryos are transferred back to the mother. To increase the live-birth rate for an IVF cycle, there is a need to select the fertilized oocytes or the embryos with better quality to be transferred back to the mother.

### SUMMARY OF CERTAIN INVENTIVE ASPECTS

The systems, methods and devices of this disclosure each have several innovative embodiments, no single one of which is solely responsible for all of the desirable attributes disclosed herein. Details of one or more implementations of the subject matter described in this specification are set forth in the accompanying drawings and the description below.

In some aspects, the techniques described herein relate to a method implemented by a system of one or more computers, the method including: applying, through a micropipette, pressure that is generated by a pressure generating device to an oocyte; obtaining a plurality of images which form an image sequence associated with a time period, the image sequence depicting the oocyte and a portion of the micropipette applying the pressure to the oocyte, wherein individual images are associated with individual pressure values applied to the oocyte at respective times of image capture; identifying, via a segmentation model, objects associated with the oocyte; determining, based at least in part on geometry measurements of the objects associated with the oocyte, features associated with the oocyte, the features including morphological features indicative of measurements of the oocyte during the time period and an aspiration depth of the oocyte into the portion of the micropipette applying the pressure to the oocyte; and generating, via a machine learning model based on input including the aspiration depth, an oocyte grade, wherein the oocyte grade is indicative of at least a likelihood of the oocyte developing into a usable blastocyst.

In some aspects, the techniques described herein relate to a method, wherein the pressure is between −0.5 psi to 0.5 psi.

In some aspects, the techniques described herein relate to a method, wherein the pressure is applied on the oocyte for 0.5 seconds to 10 seconds.

In some aspects, the techniques described herein relate to a method, wherein the pressure is generated by the pressure generating device according to a two-step pressure profile or a continued pressure profile.

In some aspects, the techniques described herein relate to a method, wherein an inner of the micropipette is between 25 microns to 100 microns.

In some aspects, the techniques described herein relate to a method, wherein the objects associated with the oocyte include at least one of a zona pellucida of the oocyte, perivitelline space of the oocyte, a first polar body of the oocyte, a cytoplasm of the oocyte, and a bounding box associated with the portion of the micropipette applying the pressure to the oocyte.

In some aspects, the techniques described herein relate to a method, wherein the morphological features associated with the oocyte include at least one of an ellipticity of the first polar body, a thickness of the zona pellucida, a diameter of the oocyte, an area of the cytoplasm, a compactness of the cytoplasm, a circularity of the cytoplasm, and a ratio between the area of the cytoplasm and a total area of the cytoplasm and the perivitelline space.

In some aspects, the techniques described herein relate to a method, wherein the segmentation model includes a U-net, and the machine learning model includes a regression model.

In some aspects, the techniques described herein relate to a method, wherein applying the pressure to the oocyte increases a probability of the oocyte forming a blastocyst.

In some aspects, the techniques described herein relate to a method implemented by a system of one or more computers for increasing a probability of an oocyte forming a blastocyst, the method including: applying, through a micropipette, pressure that is generated by a pressure generating device to an oocyte, wherein applying the pressure to the oocyte increases the probability of the oocyte forming the blastocyst.

In some aspects, the techniques described herein relate to a method, wherein the pressure generating device includes: a tank having a chamber therein, and including an opening and a communication port each of which is in fluid communication with said chamber; a deformable membrane which is disposed to seal said opening, and which is deformable between a flat state and a deformed state; a driving device which is mounted to said tank and which includes a motor body and a driving shaft, said driving shaft having a first shaft end that is disposed to confront said deformable membrane, said driving shaft being driven by said motor body to move between a distal position, where said first shaft end is distal from said motor body, and a proximate position, where said first shaft end is proximate to said motor body; and a connection unit configured to couple said first shaft end with said deformable membrane and to permit said deformable membrane to be driven by said driving shaft to transform between the flat state, where said first shaft end is in one of a distal portion and a proximate portion, and the deformed state, where said first shaft end is in the other one of the distal position and the proximate position, such that a predetermined pressure is generated through said communication port when said deformable membrane is moved from one of the flat state and the deformed state to the other one of the flat state and the deformed state.

In some aspects, the techniques described herein relate to a method, further including: obtaining a plurality of images which form an image sequence associated with a time period, the image sequence depicting the oocyte and a portion of the micropipette applying the pressure to the oocyte, wherein individual images are associated with individual pressure values applied to the oocyte at respective times of image capture; identifying, via a segmentation model, objects associated with the oocyte; determining, based at least in part on geometry measurements of the objects associated with the oocyte, features associated with the oocyte, the features including morphological features indicative of measurements of the oocyte during the time period and an aspiration depth of the oocyte into the portion of the micropipette applying the pressure to the oocyte; and generating, via a machine learning model based on input including the aspiration depth, an oocyte grade, wherein the oocyte grade is indicative of at least a likelihood of the oocyte developing into a usable blastocyst.

In some aspects, the techniques described herein relate to a method, wherein the pressure is between −0.5 psi to 0.5 psi.

In some aspects, the techniques described herein relate to a method, wherein the pressure is applied on the oocyte for 0.5 seconds to 10 seconds.

In some aspects, the techniques described herein relate to a method, wherein the pressure is generated by the pressure generating device according to a two-step pressure profile or a continued pressure profile.

In some aspects, the techniques described herein relate to a method, wherein an inner of the micropipette is between 25 microns to 100 microns.

In some aspects, the techniques described herein relate to a system including one or more processors and non-transitory computer storage media storing instructions that when executed by the one or more processors, cause the one or more processors to: apply, through a micropipette, pressure that is generated by a pressure generating device to an oocyte, wherein applying the pressure to the oocyte increases a probability of the oocyte forming a blastocyst.

In some aspects, the techniques described herein relate to a system, wherein the instructions further cause the one or more processors to: obtain a plurality of images which form an image sequence associated with a time period, the image sequence depicting the oocyte and a portion of the micropipette applying the pressure to the oocyte, wherein individual images are associated with individual pressure values applied to the oocyte at respective times of image capture; identify, via a segmentation model, objects associated with the oocyte; determine, based at least in part on geometry measurements of the objects associated with the oocyte, features associated with the oocyte, the features including morphological features indicative of measurements of the oocyte during the time period and an aspiration depth of the oocyte into the portion of the micropipette applying the pressure to the oocyte; and generate, via a machine learning model based on input including the aspiration depth, an oocyte grade, wherein the oocyte grade is indicative of at least a likelihood of the oocyte developing into a usable blastocyst.

In some aspects, the techniques described herein relate to a system, wherein the pressure generating device includes: a tank having a chamber therein, and including an opening and a communication port each of which is in fluid communication with said chamber; a deformable membrane which is disposed to seal said opening, and which is deformable between a flat state and a deformed state; a driving device which is mounted to said tank and which includes a motor body and a driving shaft, said driving shaft having a first shaft end that is disposed to confront said deformable membrane, said driving shaft being driven by said motor body to move between a distal position, where said first shaft end is distal from said motor body, and a proximate position, where said first shaft end is proximate to said motor body; and a connection unit configured to couple said first shaft end with said deformable membrane and to permit said deformable membrane to be driven by said driving shaft to transform between the flat state, where said first shaft end is in one of a distal portion and a proximate portion, and the deformed state, where said first shaft end is in the other one of the distal position and the proximate position, such that a predetermined pressure is generated through said communication port when said deformable membrane is moved from one of the flat state and the deformed state to the other one of the flat state and the deformed state.

In some aspects, the techniques described herein relate to a system, wherein the pressure is applied to the oocyte for 0.5 seconds to 10 seconds.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a schematic view illustrating a pressure generating device according to a first embodiment of the disclosure.

FIG. 2 is a schematic view of the pressure generating device similar to FIG. 1 but in a pressure increasing state.

FIG. 3 is a schematic view illustrating a pressure generating device according to a second embodiment of the disclosure.

FIG. 4 is schematic view of the pressure generating device which is similar to FIG. 3 but in a pressure decreasing state.

FIG. 5 is a perspective view illustrating a pressure generating device according to a third embodiment of the disclosure.

FIG. 6 is an exploded perspective view illustrating some elements of the pressure generating device shown in FIG. 5.

FIG. 7 is an exploded perspective view illustrating a side casing of a tank, a deformable membrane, and a connection unit in the pressure generating device shown in FIG. 5.

FIG. 8 is an exploded perspective view illustrating the side casing of the tank, the deformable membrane, and the connection unit which is similar to FIG. 7 but in an opposite direction.

FIG. 9 is an exploded perspective view illustrating a drive device, a connection sleeve of the connection unit, and a flag piece in the pressure generating device shown in FIG. 5.

FIG. 10 is a schematic view illustrating a major part of a detecting system according to an embodiment of the disclosure.

FIG. 11 is a schematic view illustrating a microscope of the detecting system according to an embodiment of the disclosure.

FIG. 12 is a graph illustrating a testing process for determining quality of a test sample in accordance with some embodiments.

FIGS. 13 and 14 are two microscope images respectively illustrating two states of an oocyte before and after a predetermined negative pressure is provided by the pressure generating device of the detecting system;

FIG. 15 is a graph illustrating another testing process for determining quality of a test sample in accordance with some embodiments.

FIGS. 16A and 16B illustrate an example implementation of applying pressure on an oocyte to increase a probability of the oocyte forming a blastocyst and using an oocyte analysis system for generating oocyte grades in accordance with some embodiments of the present disclosure.

FIG. 17 is a flowchart of an example process for applying pressure on an oocyte to increase a probability of the oocyte forming a blastocyst and generating an oocyte grade for the oocyte.

FIG. 18 illustrates a general architecture of an example oocyte analysis system in accordance with some embodiments of the present disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

It should be noted that the drawings, which are for illustrative purposes only, are not drawn to scale, and are not intended to represent the actual sizes or actual relative sizes of the components of the device or the system in this disclosure.

### Example Pressure Generating Device and Detecting System

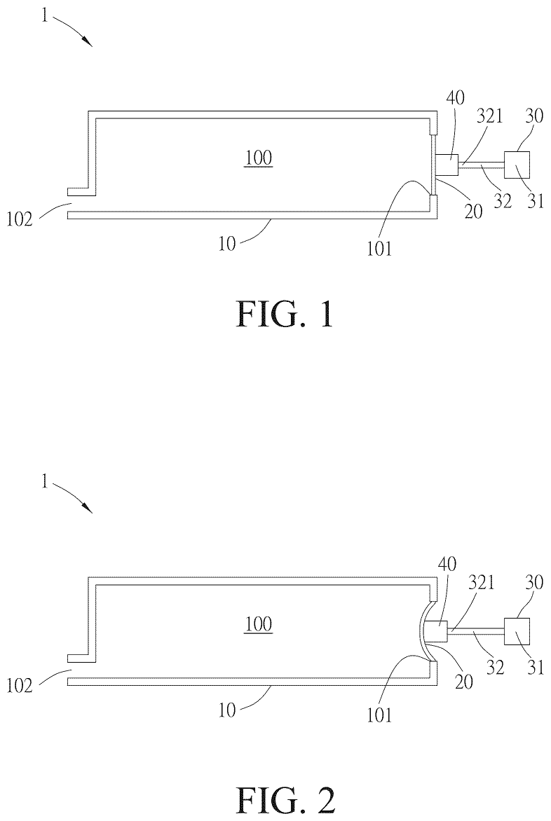

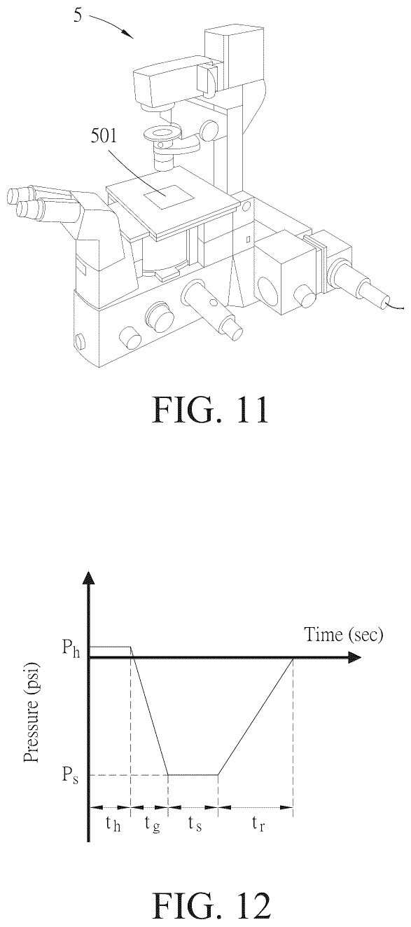

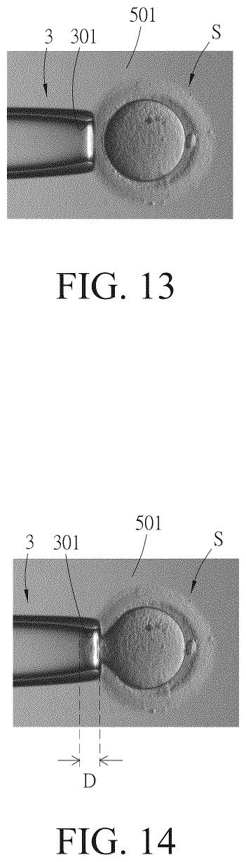

FIGS. 1 and 2 each shows a pressure generating device 1 according to a first embodiment of the disclosure. The pressure generating device 1 is used for generating a pressure to a test sample(S) through a micropipette 3 (see FIGS. 13 and 14). The test sample(S) may be an oocyte (e.g., a mammalian oocyte which may be fertilized or unfertilized) or an embryo (e.g., a mammalian embryo). Based on an aspiration depth (D) of the test sample(S) (see FIG. 14) and other factors (such as morphologies observed using a microscope 5 shown in FIG. 11), the quality of the test sample can be determined.

The pressure generating device 1 includes a tank 10, a deformable membrane 20, a driving device 30, and a connection unit 40.

The tank 10 has a chamber 100 therein, and includes an opening 101 and a communication port 102 each of which is in fluid communication with the chamber 100. The deformable membrane 20 is disposed to seal the opening 101, and is deformable between a flat state and a deformed state. The driving device 30 may be mounted to the tank 10 through any possible elements, and includes a motor body 31 and a driving shaft 32. The driving shaft 32 has a first shaft end 321 that is disposed to confront the deformable membrane 20. The driving shaft 32 is driven by the motor body 31 to move between a distal position, where the first shaft end 321 is distal from the motor body 31, and a proximate position, where the first shaft end 321 is proximate to the motor body 31. The connection unit 40 is configured to couple the first shaft end 321 with the deformable membrane 20 and to permit the deformable membrane 20 to be driven by the driving shaft 32 to transform between the flat state and the deformed state. In the flat state, the first shaft end 321 is in one of the distal portion and the proximate portion, and in the deformed state, the first shaft end 321 is in the other one of the distal position and the proximate position, such that a predetermined pressure is generated through the communication port 102 when the deformable membrane 20 is moved from one of the flat state and the deformed state to the other one of the flat state and the deformed state.

In the first embodiment, in the flat state, as shown in FIG. 1, the first shaft end 321 is in the proximate position; while in the deformed state, as shown in FIG. 2, the first shaft end 321 is in the distal position. As such, a predetermined pressure is generated through the communication port 102 when the deformable membrane 20 is moved from one of the flat state and the deformed state to the other one of the flat state and the deformed state.

In the first embodiment, in response to movement of the driving shaft 32 from the proximate position (see FIG. 1) to the distal position (see FIG. 2), the deformable membrane 20 is forced by the connection unit 40 to be deformed inwardly so as to convert the deformable membrane 20 from the flat state into the deformed state. With the deformation of the deformable membrane 20, the chamber 100 is converted into a pressure increasing state. In this case, once the deformable membrane 20 returns back to the flat state from the deformed state, the predetermined pressure can be generated from the communication port 102.

In some embodiments, the tank 10 may be made of metals, alloys, plastics, other suitable air-impermeable rigid materials, or combinations thereof. In some embodiments, the deformable membrane 20 may be made of silicone, polymer, fabric, any suitable air-impermeable flexible materials, or combinations thereof.

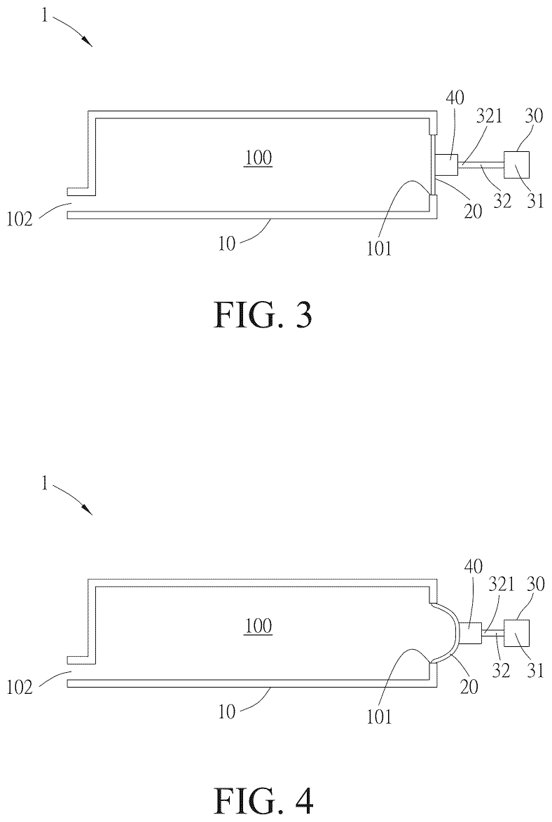

FIGS. 3 and 4 illustrate a pressure generating device 1 according to a second embodiment of the disclosure. The second embodiment is similar to the first embodiment, except that in the second embodiment, the deformable membrane 20 is deformed outwardly when in the deformed state. To be specific, in the second embodiment, the connection unit 40 is secured to the deformable membrane 20. When the driving shaft 32 is in the distal position, the deformable membrane 20 can be kept at the flat state. In response to movement of the driving shaft 32 to the proximate position (see FIG. 4) from the distal position (see FIG. 3), the deformable membrane 20 is pulled by the connection unit 40 to be deformed outwardly so as to convert the deformable membrane 20 from the flat state into the deformed state. With the deformation of the deformable membrane 20, the chamber 100 is converted into a pressure reducing state, and thus the predetermined pressure can be generated from the communication port 102.

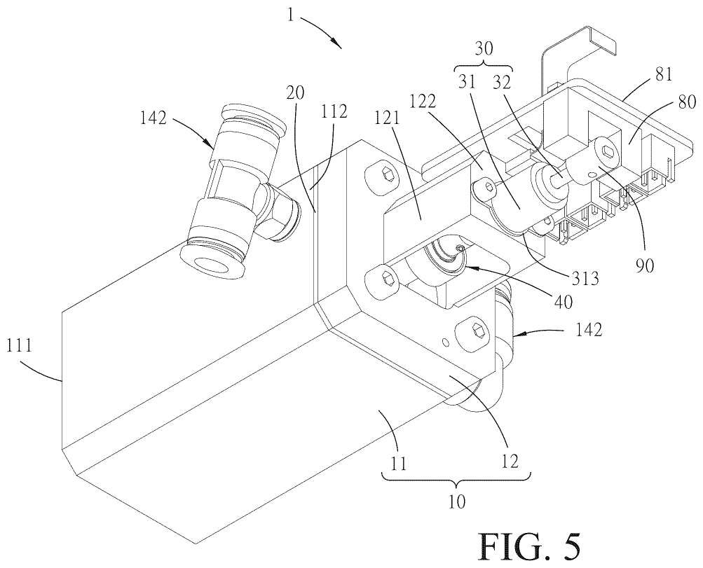

FIGS. 5 to 9 illustrate a pressure generating device 1 according to a third embodiment of the disclosure. The third embodiment is similar to the first or second embodiment, except that in the third embodiment, the tank 10, the deformable membrane 20, the driving device 30, and the connection unit 40 are shown in detail.

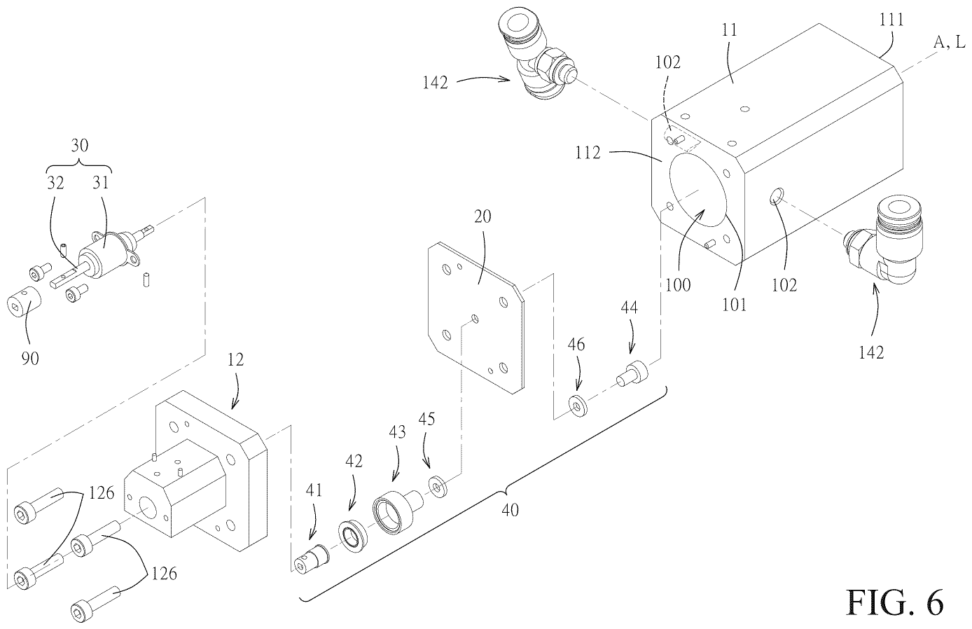

As shown in FIGS. 5 and 6, the tank 10 includes a main casing 11 and a side casing 12. The main casing 11 has the chamber 100 therein, and includes the opening 101 and the communication port 102. The side casing 12 is detachably mounted to the main casing 11 such that the deformable membrane 20 is secured between the main casing 11 and the side casing 12 to thereby seal the opening 101.

In some embodiments, the main casing 11 extends along a longitudinal axis (L) to terminate at a closed end 111 and a connection end 112 formed with the opening 101. In some embodiments, the main casing 11 is formed with two of the communication ports 102.

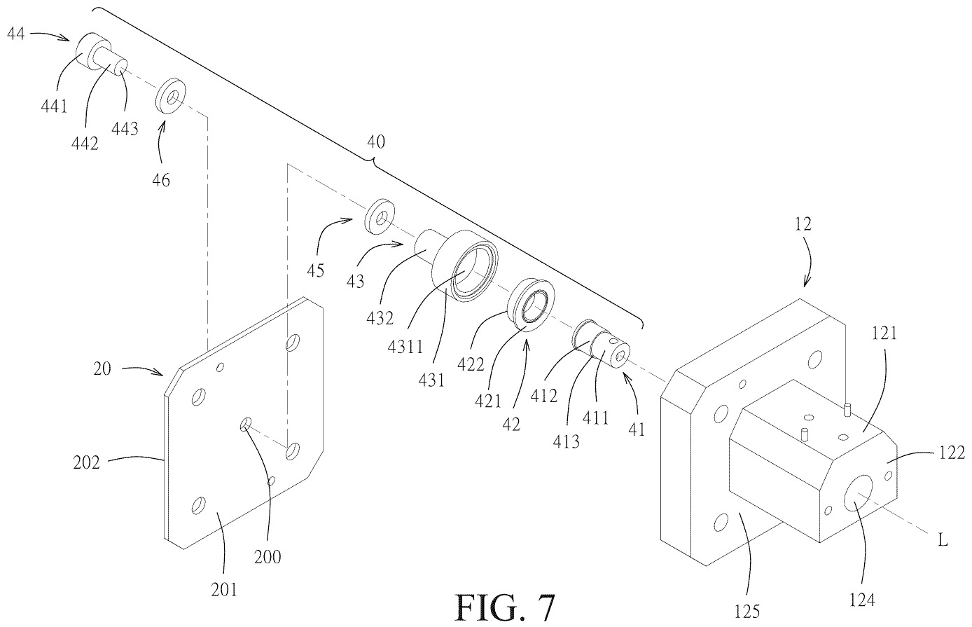

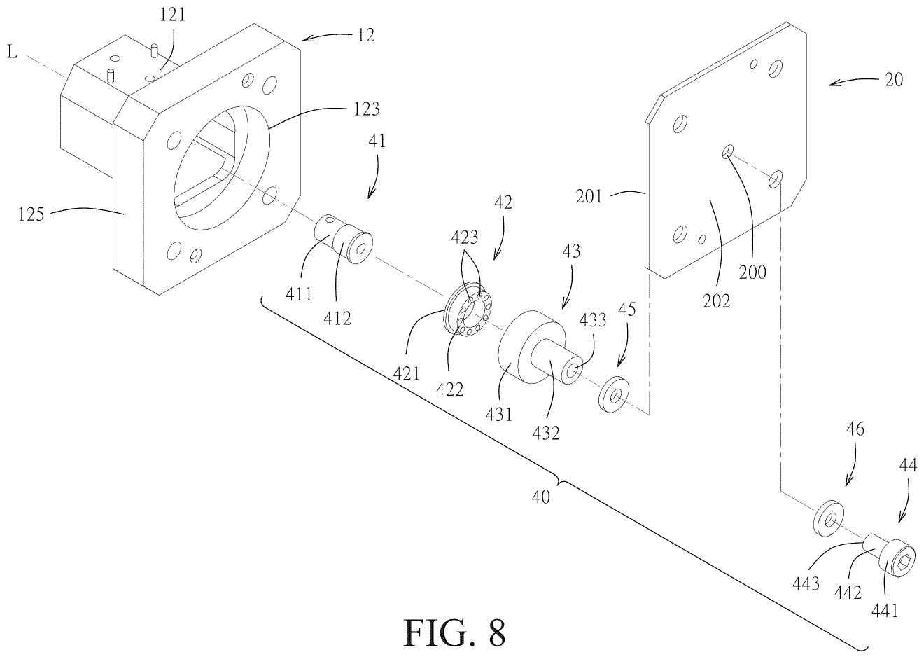

In some embodiments, referring to FIGS. 7 and 8, the side casing 12 includes a side casing body 121 and a flange body 125. The side casing body 121 has a distal end 122 and a proximal end 123 opposite to the distal end 122, and is formed with a passage 124 extending from the distal end 122 to the proximal end 123. The flange body 125 extends radially and outwardly from the proximate end 123 of the side casing body 121, and is detachably mounted to the main casing 11. In some embodiments, the deformable membrane 20 is kept at the flat state by a retaining force provided between the main casing 11 and the side casing 12.

In some embodiments, the tank 10 further includes two connection stems 142. Each of the connection stems 142 has an end that is inserted into a respective one of the communication ports 102. As such, the chamber 100 can be in fluid communication with a connection tube 4 (see FIGS. 1 to 4 and 10) through one of the connection stems 142. Each of the connection stems 142 may have a valve (not shown) mounted thereon, and the valve is switchable between a first state, where the one of the connection stems 142 is in an open state to permit the chamber 100 to be in fluid communication with the connection tube 4, and a second state, where the one of the connection stems 142 is in a closed state to block the communication between the chamber 100 and the connection tube 4.

In some embodiments, a plurality of fasteners 126 are provided to pass through the flange body 125 and the deformable membrane 20, and extend into the connection end 112 of the main casing 11 so as to fasten the side casing 12 to the main casing 11.

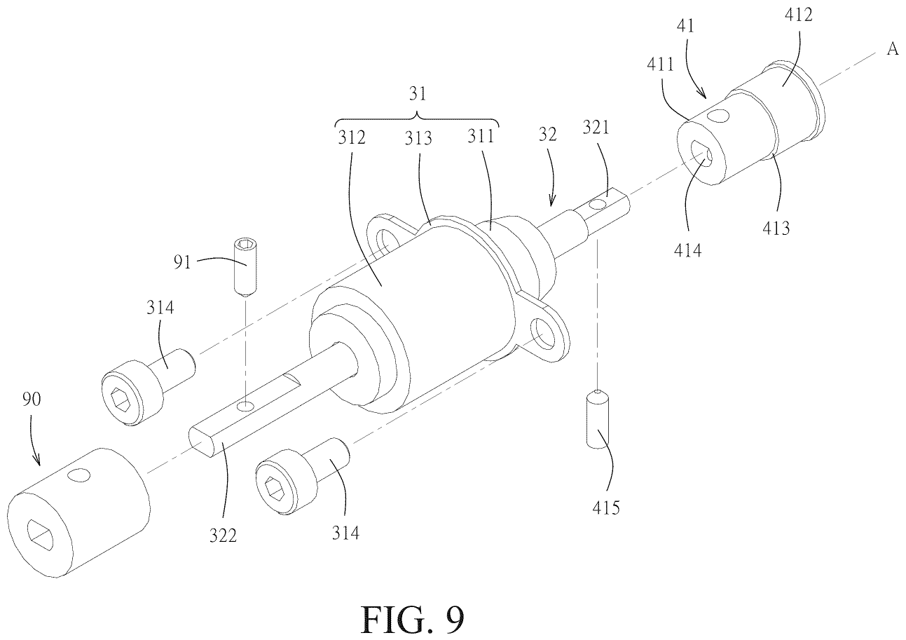

As shown in FIGS. 5 and 9, the driving device 30 is mounted to the tank 10. In some embodiments, the motor body 31 is immovably mounted to the side casing 12, and the driving shaft 32 extends along a shaft axis (A) through the motor body 31, and is driven by the motor body 31 to move toward or away from the deformable membrane 20 along the shaft axis (A) so as to transform the deformable membrane 20 between the flat state and the deformed state. In some embodiments, the shaft axis (A) may be in alignment with the longitudinal axis (L). In some embodiments, the driving device 30 is a linear motor, and the driving shaft 32, when being driven to move along the shaft axis (A), is not rotatable about the shaft axis (A). In some other embodiments, the driving device 30 is a stepper motor, and the driving shaft 32, when being driven to move along the shaft axis (A), rotates about the shaft axis (A).

In some embodiments, referring to FIG. 9, the motor body 31 has an inner portion 311, an outer portion 312 and a flange portion 313. The inner portion 311 is received in the passage 124 of the side casing body 121. The outer portion 312 extends from the inner portion 311 in a direction away from the main casing 11, and is disposed outside the side casing body 121. The flange portion 313 extends radially from the outer portion 312, and is detachably secured to the distal end 122 of the side casing body 121 by fasteners 314.

In some embodiments, the driving shaft 32 further has a second shaft end 322 which is opposite to the first shaft end 321 along the shaft axis (A). The first and second shaft ends 321, 322 are located at two opposite sides of the motor body 31.

As shown in FIGS. 7 and 8, in some embodiments, the connection unit 40 includes a connection sleeve 41, a bearing sleeve 42, and a bearing cap 43.

The connection sleeve 41 is coupled on the first shaft end 321 of the driving shaft 32 so as to permit the connection sleeve 41 to move with the driving shaft 32 along the shaft axis (A). The connection sleeve 41 has a first sleeve portion 411, a second sleeve portion 412, and a shoulder surface 413. The second sleeve portion 412 has an outer dimension that is larger than an outer dimension of the first sleeve portion 411. The shoulder surface 413 is located between the first sleeve portion 411 and the second sleeve portion 412. In some embodiments, as shown in FIG. 9, the connection sleeve 41 is formed with a through bore 414, and the first shaft end 321 is non-rotatably secured in the through bore 414 by a fastener 415.

The bearing sleeve 42 is fittingly sleeved on the second sleeve portion 412 so as to permit the bearing sleeve 42 to move with the connection sleeve 41 along the shaft axis (A), and is disposed between the connection sleeve 41 and the bearing cap 43. The bearing sleeve 42 has a first abutment end 421 and a second abutment end 422 opposite to the first abutment end 421. In some embodiments, a plurality of bearing balls 423 are formed in the second abutment end 422 and are angularly displaced from each other about the shaft axis (A).

The bearing cap 43 has an engaging portion 431 and a mounted portion 432. The engaging portion 431 is formed with a recess 4311 that is configured to engage with the second abutment end 422 so as to permit the bearing cap 43 to move with the bearing sleeve 42 along the shaft axis (A). The mounted portion 432 is opposite to the engaging portion 431 in the shaft axis (A), and is secured to a first surface 201 of the deformable membrane 20 so as to transmit a force from the driving shaft 32 to the deformable membrane 20.

In some embodiments, the deformable membrane 20 is formed with a through hole 200, and the connection unit 40 further includes a fastener 44. The fastener 44 has an enlarged head 441 and a fastening rod 442. The enlarged head 441 is disposed on a second surface 202 of the deformable membrane 20 opposite to the first surface 201. The fastening rod 442 extends from the enlarged head 441 through the through hole 200 of the deformable membrane 20 to terminate at a rod end 443. The rod end 443 is fittingly engaged within a hole 433 formed in the mounted portion 432 of the bearing cap 43 so as to permit the deformable membrane 20 to be fastened to the bearing cap 43 through the fastener 44.

In some embodiments, the connection unit 40 further includes a first washer 45 and a second washer 46. The first washer 45 is disposed between the mounted portion 432 and the first surface 201 of the deformable membrane 20. The second washer 46 is disposed between the enlarged head 441 and the second surface 202 of the deformable membrane 20. In some embodiments, in order to improve transmission of a torque from the driving shaft 32 to the deformable membrane 20, each of the first and second washers 45, 46 is made of a deformable material. Examples for the deformable material may be similar to the materials suitable for forming the deformable membrane 20, and thus the details thereof are omitted for the sake of brevity.

In some embodiments, with reference to FIGS. 1, 2 and 5 to 9, the deformable membrane 20 is deformed inwardly in the deformed state. In other words, in response to movement of the driving shaft 32 from the proximate position to the distal position, the bearing cap 43 is forced by the first shaft end 321 (through the connection sleeve 41 and the bearing sleeve 42) to move toward the chamber 100 and is brought into pressing engagement with the deformable membrane 20 so as to force the deformable membrane 20 to deform into the deformed state against the retaining force provided between the main casing 11 and the side casing 12. In response to movement of the driving shaft 32 from the distal position (see FIG. 2) to the proximate position (see FIG. 1), the bearing cap 43 is brought to move away from the chamber 100 so as to permit the deformable membrane 20 to return back to the flat state by the retaining force, thereby allowing the communication port 102 (or one of the communication ports 102 shown in FIG. 6) to generate the predetermined pressure (e.g., a predetermined negative pressure).

In some other embodiments, with reference to FIGS. 3 to 9, the deformable membrane 20 is deformed outwardly in the deformed state. In this case, the driving device 30 is a linear motor, and the connection sleeve 41, the bearing sleeve 42 and the bearing cap 43 are secured to each other. In some embodiments, every two adjacent ones of the connection sleeve 41, the bearing sleeve 42 and the bearing cap 43 are fittingly engaged with each other. In some other embodiments, an adhesive agent may be additionally applied between every two adjacent ones of the connection sleeve 41, the bearing sleeve 42 and the bearing cap 43. To be specific, in response to movement of the driving shaft 32 from the distal position (see FIG. 3) to the proximate position (see FIG. 4), the deformable membrane 20 is pulled by the connection unit 40 so as to deform outwardly into the deformed state against the retaining force between the main casing 11 and the side casing 12, thereby generating the predetermined pressure (e.g., a predetermined negative pressure) from the communication port 102 or one of the communication ports 102 shown in FIG. 6. In response to movement of the driving shaft 32 from the proximate position to the distal position, the connection unit 40 moves toward the chamber 100 so as to permit the deformable membrane 20 to return back to the flat state by the retaining force.

In some embodiments, the deformable membrane 20 has a hardness ranging from 0 Shore A to 100 Shore A, e.g., from 0 Shore A to 10 Shore A, from 5 Shore A to 15 Shore A, from 10 shore A to 20 Shore A, from 15 Shore A to 25 Shore A, from 20 Shore A to 30 Shore A, from 25 Shore A to 35 Shore A, from 30 Shore A to 40 Shore A, from 35 Shore A to 45 Shore A, from 40 Shore A to 50 Shore A, from 45 Shore A to 55 Shore A, from 50 Shore A to 60 Shore A, from 55 Shore A to 65 Shore A, from 60 Shore A to 70 Shore A, from 65 Shore A to 75 Shore A, from 70 Shore A to 80 Shore A, from 75 Shore A to 85 Shore A, from 80 Shore A to 90 Shore A, from 85 Shore A to 95 Shore A, or from 90 Shore A to 100 Shore A. The driving device 30 is configured to provide a thrust which ranges from 100 g to 1000 g, e.g., from 100 g to 200 g, from 150 g to 250 g, from 200 g to 300 g, from 250 g to 350 g, from 300 g to 400 g, from 350 g to 450 g, from 400 g to 500 g, from 450 g to 550 g, from 500 g to 600 g, from 550 g to 650 g, from 600 g to 700 g, from 650 g to 750 g, from 700 g to 800 g, from 750 g to 850 g, from 800 g to 900 g, from 850 g to 950 g, or from 900 g to 1000 g. A stroke of the driving device 30 ranges from 3 mm to 50 mm, e.g., from 3 mm to 15 mm, from 9 mm to 21 mm, from 15 mm to 27 mm, from 21 mm to 33 mm, from 27 mm to 39 mm, from 33 mm to 45 mm, or from 39 mm to 50 mm.

The stroke and the thrust of the driving device 30 are determined based on a required degree of deformation of the deformable membrane 20 and the hardness of the deformable membrane 20, respectively. When the stroke required for the driving device 30 is relatively long and the hardness of the deformable membrane 20 is relatively small, operation of the driving device 30 is relatively easier (i.e., the thrust required for the driving device 30 is relatively small). When the hardness of the deformable membrane 20 becomes larger, the thrust required for the driving device 30 will be increased if the stroke required for the driving device 30 is not reduced.

In some embodiments, a volume of the main casing 11 ranges from 1 cm3 to 50 cm3, e.g., from 1 cm3 to 10 cm3, from 5 cm3 to 15 cm3, from 10 cm3 to 20 cm3, from 15 cm3 to 25 cm3, from 20 cm3 to 30 cm3, from 25 cm3 to 35 cm3, from 30 cm3 to 40 cm3, from 35 cm3 to 45 cm3, or from 40 cm3 to 50 cm3.

In some embodiment, the pressure generating device 1 further includes a photo sensor 80 which is immovable relative to the tank 10 so as to permit the photo sensor 80 to detect a displacement of the driving shaft 32. In some embodiments, a circuit board 81 is mounted to the side casing body 121, and the photo sensor 80 is mounted on the circuit board 81 and is electrically connected to a circuit of the circuit board 81.

In some embodiment, referring to FIGS. 5 and 9, the pressure generating device 1 further includes a flag piece 90 which is mounted on the second shaft end 322 of the driving shaft 32 so as to permit the flag piece 90 to move with the driving shaft 32, and which has a plurality of detectable positions to be detected by the photo sensor 80, thereby determining the displacement of the driving shaft 32. In some embodiments, the flag piece 90 is mounted to the second shaft end 322 by a fastener 91.

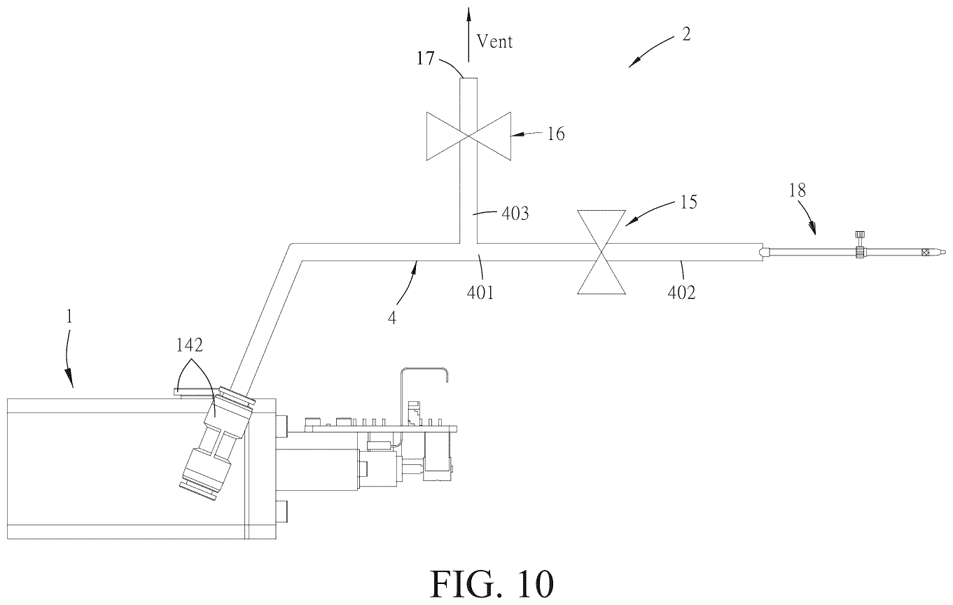

FIG. 10 illustrates a major part of a detecting system 2 for detecting quality of the test sample (S, see FIGS. 13 and 14) according to an embodiment of the disclosure. The detecting system 2 includes the pressure generating device 1 (in which one of the connection stems 142 is in the open state, the other one of the connection stems 142 is in the closed state), the micropipette 3 (see FIGS. 13 and 14) for sucking the test sample(S), and the connecting tube 4 disposed to connect one of the communication ports 102 of the tank 10 (through the one of the connection stems 142) to the micropipette 3 for applying the predetermined pressure (e.g., a predetermined negative pressure) from the pressure generating device 1 to the test sample(S). In some other embodiments, the detecting system 2 may include the pressure generating device 1 (in which both the connection stems 142 are in the open state), two of the micropipettes 3 (only one of which is shown in FIGS. 13 and 14) and two of the connecting tubes 4 (only one of which is shown in FIG. 10) so that the predetermined pressure from the pressure generating device 1 can be applied to two test samples(S) (only one of which is shown) through the connection stems 142, respectively. In the followings, each of the communication port 102, the connection stem 142, the micropipette 3, the connection tube 4 and so on is described in a singular form.

In some embodiments, an inner diameter of the micropipette 3 ranges from about 10 microns to about 100 microns (e.g., about 40 microns to about 70 microns).

In some embodiments, by the pressure generating device 1, a pressure ranging from about 0.5 psi to about −0.5 psi is generated at a suction port 301 of the micropipette 3. For example, the pressure generated at the suction port 301 of the micropipette 3 may varied from about −0.03 psi to about −0.5 psi, from about 0.03 psi to about −0.5 psi, from about 0.03 psi to about 0.5 psi, or from about −0.03 psi to about 0.5 psi.

In some embodiments, referring to FIGS. 10, 11, 13 and 14, the detecting system 2 further includes a microscope 5 and an injection holder 18. The microscope 5 is used for monitoring the test sample(S) and the suction port 301 of the micropipette 3. To be specific, the test sample(S) is placed on a dish 501 of the microscope 5 and the injection holder 18 is connected between the connecting tube 4 and the micropipette 3. In operation, the suction port 301 of the micropipette 3 is retained by the injection holder 18 to be disposed on the dish 501 of the microscope 5, so as to facilitate the test sample(S) on the dish 501 to be retained and/or sucked by the suction port 301 of the micropipette 3.

In some embodiments, the connecting tube 4 includes a first tube segment 401 connected to the communication port 102 of the tank 10, and a second tube segment 402 connected to the micropipette 3 through the injection holder 18. The first and second tube segments 401, 402 are connected to each other through an adjusting valve 15 for adjusting the pressure generated at the suction port 301 of the micropipette 3. In some embodiments, the adjusting valve 15 is a solenoid valve.

In some embodiments, the detecting system 2 further includes a branch tube 403 which is connected to the first tube segment 401, and which has the venting port 17 opposite to the first tube segment 401. In addition, a venting valve 16 is coupled to the branch tube 403 to control a fluid communication between the chamber 100 and the venting port 17. To be specific, when the venting valve 16 is opened, the venting port 17 is in fluid communication with the chamber 100, and when the venting valve 16 is fully closed, the venting port 17 is prevented from being in fluid communication with the chamber 100. Therefore, when the venting valve 16 is opened, the pressure inside the chamber 100 is permitted to return to an atmospheric pressure. In some embodiments, the venting valve 16 is a solenoid valve.

FIG. 12 is a graph illustrating a testing process for determining the quality of the test sample(S) in accordance with some embodiments. The testing process shown in FIG. 12 is described with reference to FIGS. 10, 13 and 14. During the testing process illustrated in FIG. 12, the adjusting valve 15 is always opened.

Referring to FIGS. 10 and 12, in the beginning, the venting valve 16 is fully closed, and the pressure inside the chamber 100 is adjusted to and kept at an initial pressure (e.g., a slightly positive or negative pressure), and the position of the actuating arm 40 is also moved to permit the deformable membrane 20 to be kept at a home state. For example, in the case that the deformable membrane 20 is deformed inwardly (e.g., the embodiment shown in FIGS. 1 and 2), the home state of the deformable membrane 20 is the deformed state (see FIG. 2); while in the case that the deformable membrane 20 is deformed outwardly (e.g., the embodiment shown in FIGS. 3 and 4), the home state of the deformable membrane 20 is the flat state (see FIG. 3). Due to the initial pressure of the chamber 100, as shown in FIG. 12, a slightly positive pressure (Pₕ) (e.g., about 0.01±10% psi) is generated at the suction port 301 of the micropipette 3 and kept for a time period (tₕ), and thus the test sample(S) is retained by the suction port 301 of the micropipette 3 and is prevented from being sucked into the micropipette 3 (see FIG. 13). Then, by actuating the driving device 30 to cause transformation of the deformable membrane 20, a pressure from the communication port 102 is continuously reduced for a time period (t_{g}) (e.g., 2±10% seconds) so as to reach the predetermined pressure (e.g., a predetermined negative pressure), and meanwhile, a suction pressure generated at the suction port 301 of the micropipette 3 is gradually decreased to a stress pressure (Pₛ) (e.g., −0.1±10% psi). The stress pressure (Pₛ) lasts for a time period (tₛ) (e.g., 1±10% second) and at this stage, as shown in a microscope image in FIG. 14, an aspiration depth (D) of the test sample(S) is determined using a computer program (not shown). Finally, the driving device 30 is further actuated to continuously increase the pressure inside the chamber 100 for a time period (tᵣ), thereby returning the deformable membrane 20 to the home state. Thereafter, the venting valve 16 is opened to permit the pressure inside the chamber 100 to return to the atmospheric pressure through the venting port 17. Please note that the pressure inside the chamber 100 is increased gradually so as to avoid blowing away the test sample(S) from the micropipette 3. In some other embodiments, before reduction to reach the stress pressure (Pₛ), the pressure (Pₕ) generated at the suction port 301 of the micropipette 3 may be kept at a slightly negative pressure (e.g., −0.01±10% psi) for a time period (e.g., 2±10% seconds) and for sticking the test sample(S).

During the testing process shown in FIG. 12, the driving shaft 32 can be driven to move between the proximate and distal positions.

In some embodiments in which the deformable membrane 20 is deformed inwardly in the deformed state (e.g., the embodiment shown in FIGS. 1 and 2), (i) at the time period (tₕ), the driving shaft 32 is in the distal position and the deformable membrane 20 is in the deformed state (see FIG. 2), and (ii) at the time period (tₛ), the driving shaft 32 is in the proximate position, the deformable membrane 20 is in the flat state (see FIG. 1).

In some other embodiments in which the deformable membrane 20 is deformed outward in the deformed state (e.g., the embodiment shown in FIGS. 3 and 4), (i) at the time period (tₕ), the driving shaft 32 is in the distal position and the deformable membrane 20 is in the flat state (see FIG. 3), and (ii) at the time period (tₛ), the driving shaft 32 is in the proximate position and the deformable membrane 20 is in the deformed state (see FIG. 4).

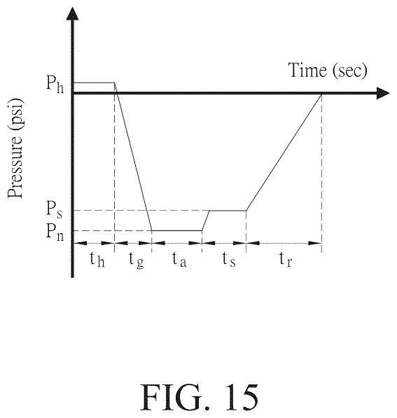

FIG. 15 is a graph illustrating another testing process for determining the quality of the test sample(S) in accordance with some embodiments. The testing process shown in FIG. 15 is described with reference to FIGS. 10, 13 and 14. During the testing process illustrated in FIG. 15, the adjusting valve 15 is switched to adjust the pressure generated at the suction port 301 of the micropipette 3.

Referring to FIGS. 10 and 15, in the beginning, the venting valve 16 is fully closed, the adjusting valve 15 is fully closed, and the pressure inside the chamber 100 is adjusted to and kept at an initial pressure (e.g., a lower positive pressure), and the position of the driving shaft 32 is also moved to permit the deformable membrane 20 to be kept at a home state. For example, in the case that the deformable membrane 20 is deformed inwardly (e.g., the embodiment shown in FIGS. 1 and 2), the home state of the deformable membrane 20 is the deformed state (see FIG. 2); while in the case that the deformable membrane 20 is deformed outwardly (e.g., the embodiment shown in FIGS. 3 and 4), the home state of the deformable membrane 20 is the flat state (see FIG. 3). Due to the initial pressure of the chamber 100, by opening the adjusting valve 15, a slightly positive pressure (Pₕ) (e.g., about 0.01±10% psi) is generated at the suction port 301 of the micropipette 3 and kept for a time period (tₕ) (see FIG. 15), and thus the test sample(S) is retained by the suction port 301 of the micropipette 3 and is prevented from being sucked into the micropipette 3 (see FIG. 13). Then, the adjusting valve 15 is closed, and by actuating the driving device 30 to cause transformation of the deformable membrane 20, a pressure inside the chamber 100 is continuously reduced for a time period (t_{g}) (e.g., 3±10% seconds) so as to reach a preset suction pressure (Pₙ) (e.g., −0.13±10% psi). The preset suction pressure (Pₙ) is kept for a time period (tₐ) (e.g., 2±10% seconds). Then, during a time period (tₛ) (e.g., 4±10% second), by slightly or fully opening the adjusting valve 15, the pressure inside the chamber 100 is slightly increased to and kept at a stress pressure (Pₛ) (e.g., −0.1±10% psi) and in meanwhile, the suction pressure generated at the suction port 301 of the micropipette 3 is substantially equal to the stress pressure (Pₛ). At this stage (i.e., the test sample(S) under the stress pressure (Pₛ)), as shown in a microscope image in FIG. 14, an aspiration depth (D) of the test sample(S) is determined using the computer program (not shown). Finally, the driving device 30 is further actuated to continuously increase the pressure inside the chamber 100 for a time period (tᵣ), thereby returning the deformable membrane 20 to the home state. Thereafter, the venting valve 16 is opened to permit the pressure inside the chamber 100 to return to the atmospheric pressure through the venting port 17. Please note that the pressure inside the chamber 100 is increased gradually so as to avoid blowing away the test sample(S) from the micropipette 3. In some other embodiments, before reduction to reach the preset suction pressure (Pₙ), the pressure (Pₕ) generated at the suction port 301 of the micropipette 3 may be kept at a slightly negative pressure (e.g., −0.01±10% psi) for a time period (e.g., 2±10% seconds) and for sticking the test sample(S).

During the testing process shown in FIG. 15, the driving shaft 32 can be driven to move between the proximate and distal positions.

In some embodiments in which the deformable membrane 20 is deformed inwardly in the deformed state (e.g., the embodiment shown in FIGS. 1 and 2), (i) at the time period (tₕ), the driving shaft 32 is in the distal position and the deformable membrane 20 is in the deformed state (see FIG. 2), and (ii) at the time periods (tₐ, tₛ), the driving shaft 32 is in the proximate position and the deformable membrane 20 is in the flat state (see FIG. 1).

In some other embodiments in which the deformable membrane 20 is deformed outwardly in the deformed state (e.g., the embodiment shown in FIGS. 3 and 4), (i) at the time period (tₕ), the driving shaft 32 is in the distal position and the deformable membrane 20 is in the flat state (see FIG. 3), and (ii) at the time periods (tₐ, tₛ), the driving shaft 32 is in the proximate position and the deformable membrane 20 is in the deformed state (see FIG. 4).

With the provision of the detecting system 2 including the pressure generating device 1, a suction pressure can be generated at the suction port 301 of the micropipette 3 for sucking the test sample(S). Based on the aspiration depth (D) of the test sample(S), the quality of the test sample(S) may be determined. For example, a test sample(S) with a relatively smaller aspiration depth (D) may have a better quality. In addition, because the suction pressure generated using the pressure generating device 1 is relatively small, the test sample(S) is less likely to be damaged during the testing process, and the qualified test sample(S) can be transferred back to the mother for establishing a successful pregnancy.

### Example Pressure Application for Boosting Blastocyst Formation

As discussed above with respect to FIGS. 1 and 15, the pressure generating device 1 can be utilized for generating and applying a pressure (e.g., a negative pressure), through a micropipette 3, on a test sample(S) (shown in FIGS. 13 and 14) such as an oocyte (e.g., a mammalian oocyte which may be fertilized or unfertilized) or an embryo (e.g., a mammalian embryo). Based on an aspiration depth (D) of the test sample(S) shown in FIG. 14 and other factors (such as morphologies observed using a microscope 5 shown in FIG. 11), the quality of the test sample(S) can be determined.

As will be discussed below, the pressure generating device 1 can be utilized for boosting blastocyst formation by applying pressure (e.g., a negative pressure) to oocytes using a micropipette (e.g., the micropipette 3 or any other micropipette) according to some embodiments of the present disclosure. Advantageously, applying pressure to oocytes (e.g., using the pressure generating device 1 or any other pressure generating device) provides non-invasive mechanisms to improve the quality and/or developmental potential (e.g., the probability of blastocyst formation) of oocytes without harming the oocytes, and can be particularly useful for improving developmental potential of oocytes that otherwise have low probability (e.g., aging oocytes that exhibit declining qualities over the years) of forming blastocysts.

In some embodiments, a pressure generating device (e.g., the pressure generating device 1 or any other pressure generating device) can apply pressure of various pressure profiles (e.g., “two-step” pressure profile illustrated in FIG. 15, and/or “continued” pressure profile illustrated in FIG. 12), pressure ranges (e.g., various levels of pressures), and/or time ranges (e.g., various durations in which pressure is applied) through a micropipette (e.g., the micropipette 3) on oocytes to boost the probability of the oocytes developing into or forming blastocysts, thereby addressing the issues of declining oocyte quality and/or improving the success rates of in vitro fertilization (IVF) and intracytoplasmic sperm injection (ICSI) procedures.

In some examples, the pressure ranges applied by the pressure generating device through the micropipette on oocytes can be at a pressure of, of about, of at least, or of at least about, −0.5 psi, −0.4 psi, −0.3 psi, −0.2 psi, −0.15 psi, −0.14 psi, −0.13 psi, −0.12 psi, −0.11 psi, −0.1 psi, −0.05 psi, 0.05 psi, 0.1 psi, 0.2 psi, 0.3 psi, 0.4 psi, or 0.5 psi, or any range of values therebetween.

In some examples, the time ranges (e.g., a duration of time in which pressure of various pressure profiles and/or pressure ranges is applied) applied by the pressure generating device through the micropipette on oocytes can be at a time interval of, of about, of at least, or of at least about, 0.5 seconds (sec), 1.0 seconds, 2.0 seconds, 3.0 seconds, 4.0 seconds, 5.0 seconds, 6.0 seconds, 7.0 seconds, 8.0 seconds, 9.0 seconds, 10.0 seconds, or any range of values therebetween.

An example pressure applied on an oocyte can correspond to the “continued” pressure profile illustrated in FIG. 12, with a pressure of −0.13 psi applied for 2.0 seconds. More specifically, in this example, a stress pressure (Pₛ) in FIG. 12 is −0.13 psi and a time period (tₛ) in FIG. 12 is 2.0 seconds. Another example pressure applied on an oocyte can correspond to the “two-step” pressure profile illustrated in FIG. 15, with a pressure of −0.15 psi applied for 3.0 seconds. More specifically, in this example, a stress pressure (Pₛ) in FIG. 15 is −0.15 psi and a time period (tₛ) in FIG. 15 is 3.0 seconds. Advantageously, by applying pressure of various pressure profiles, pressure ranges, and/or time ranges specified above to oocytes, it is less likely to damage the oocytes and more likely to effectively activate, vitalize, and/or stimulate the oocytes to increase the probability of the oocytes developing into blastocysts.

Additionally and/or optionally, an inner diameter of the micropipette can be adjusted and can be at a length of, of about, of at least, or of at least about, 25 microns, 30 microns, 40 microns, 50 microns, 60 microns, 70 microns, 80 microns, 90 microns, 100 microns, or any range of values therebetween in some embodiments.

Table 1 illustrates example options and/or ranges associated with pressure profiles, pressure ranges, and time ranges of pressure that can be applied by a pressure generating device (e.g., the pressure generating device 1 or any other pressure generating device) through a pipette (e.g., the micropipette 3 or any other micropipette) to increase probability of blastocyst formation of an oocyte.

TABLE 1

| | Pressure Profile | Pressure Ranges | Time Ranges |
|---|---|---|---|
| Options/Ranges | continued or | −0.5 psi to | 0.5 sec to |
| | two-step | 0.5 psi | 10 sec |

As an example showing effectiveness associated with applying pressure on oocytes to boost probability of blastocyst formation, an experiment was conducted on two groups of oocytes, where oocytes in a first group (e.g., a control group) were not applied pressure for boosting blastocyst formation and oocytes in a second group (e.g., an experiment group) were applied pressure (e.g., pressure of pressure profiles, pressure ranges, and/or time ranges specified above such as “two-step” pressure profile with a pressure at −0.13 psi applied for 2.0 seconds) for boosting blastocyst formation. The experiment results show that, for oocytes in the first group, 75% fertilized, 48% formed good embryo (e.g., day 3 good embryo), and 45% formed blastocysts. For oocytes in the second group that were applied pressure for boosting blastocyst formation, 77% fertilized, 51% formed good embryo (e.g., day 3 good embryo), and 56% formed blastocysts.

### Example Oocyte Analysis System

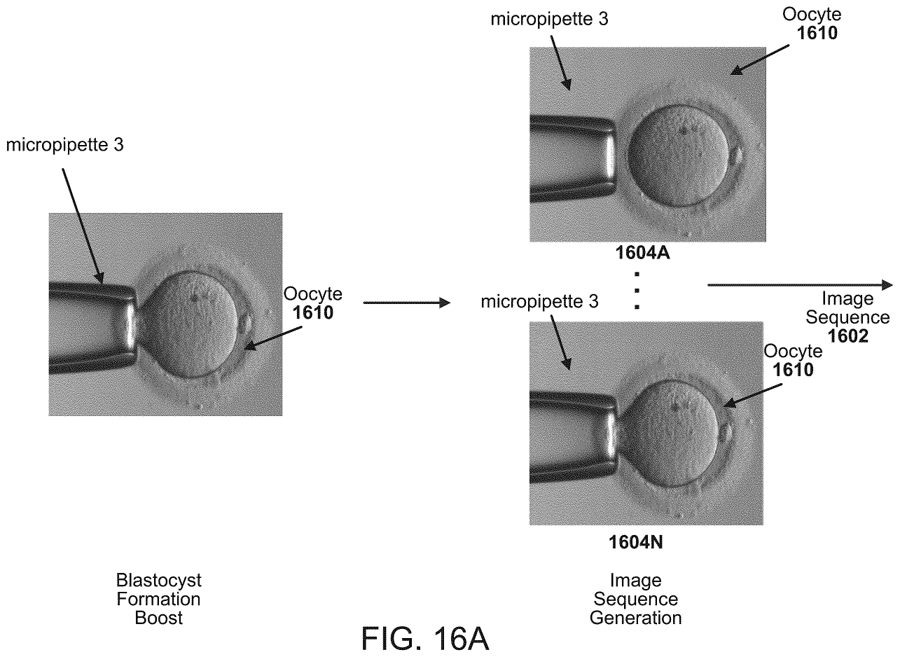

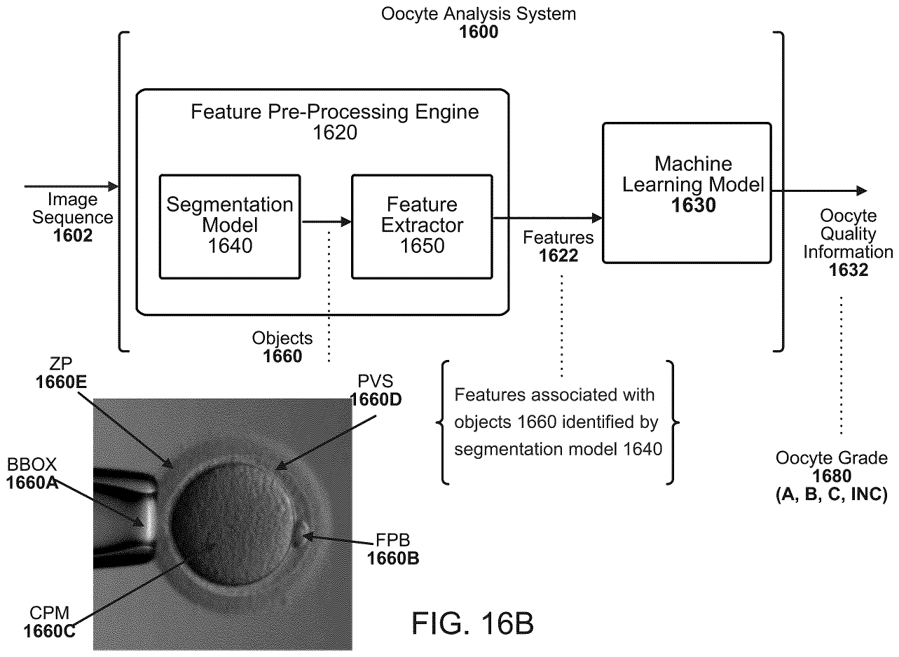

FIGS. 16A and 16B illustrate an example implementation of applying pressure on an oocyte 1610 for increasing a probability of the oocyte 1610 forming a blastocyst, and using an oocyte analysis system 1600 for generating oocyte grades in accordance with some embodiments of the present disclosure. As shown in FIG. 16A, pressure may be generated by a pressure generating device (e.g., the pressure generating device 1 and any other pressure generating device, not shown in FIG. 16A) and applied on the oocyte 1610 through the micropipette 3 for increasing probability of blastocyst formation of the oocyte 1610. The pressure generated by the pressure generating device may include any combinations of pressure profile, pressure ranges, and/or time ranges illustrated in Table 1. It should be noted that the pressure (e.g., negative pressure) generated by the pressure generating device (e.g., the pressure generating device 1) can occur and/or be used in system(s) other than the oocyte analysis system 1600 to increase probability of blastocyst formation of the oocyte 1610 in other embodiments.

Before, after, or concurrently to applying pressure on the oocyte 1610 for increasing probability of blastocyst formation of the oocyte 1610, an image sequence 1602 may be generated. More specifically, the image sequence 1602 may include multiple images (e.g., images 1604A-1604N), for which each image depicts the oocyte 1610 along with the micropipette 3. More specifically, images 1604A-1604N may form a video depicting a process of aspirating (e.g., due to the pressure applied on the oocyte 1610) the oocyte 1610 into the micropipette 3. Image 1604A can represent, for example, the first frame of the video and image 1604N can represent, for example, the last frame of the video. Image 1604A depicts the oocyte 1610 as not yet being aspirated into the micropipette 3 while image 1604N depicts at least part of the oocyte 1610 being aspirated into the micropipette 3. In some examples, the image sequence 1602 may have a frame rate of 10 Hz, 20 Hz, 70 Hz, 3000 Hz, with a total video length of 1 second, 2 seconds, 10 seconds, and so on.

In some embodiments, the micropipette 3 is a pipette that has a diameter between particular thresholds (e.g., between 10 microns (μm), 20 μm, 40 μm, 50 μm, 60 μm, 70 μm, 100 μm, and so on). The micropipette 3 may apply a negative pressure (e.g., a lower pressure inside the pipette relative to the pressure outside the pipette) on the oocyte 1610 for aspirating the oocyte 1610 into the pipette without damaging the oocyte 1610. An example pressure applied during the generation of the image sequence 1602 may include pressure between −0.01 psi to −0.5 psi. For example, the micropipette 3 may abut or otherwise be in contact with the oocyte 1610. Although the image sequence 1602 illustrates the oocyte 1610 being aspirated into the micropipette 3, in some embodiments the micropipette 3 may apply other forms of mechanical stimulus (e.g., positive pressure). In this way, different morphological responses of the oocyte 1610 may be obtained for further processing by the oocyte analysis system 1600.

As shown in FIG. 16B, the oocyte analysis system 1600 may include a feature pre-processing engine 1620 and a machine learning model 1630 for analyzing the image sequence 1602 to generate an oocyte quality information 1632. In some embodiments, the feature pre-processing engine 1620 may receive the image sequence 1602 of the oocyte 1610. Based on the image sequence 1602, the segmentation model 1640 may identify objects 1660 associated with the oocyte 1610. Based on the objects 1660, the feature extractor 1650 may determine or extract the features 1622 associated with the objects 1660 that are identified by the segmentation model 1640. Although not illustrated in FIG. 16B, the feature pre-processing engine 1620 may additionally and/or optionally utilize other information (e.g., pressure values applied on the oocyte 1610 during generation of the image sequence 1602 and/or clinical information associated with a patient from whom the oocyte 1610 was obtained as inputs for generating the features 1622). Based at least on a subset of the features 1622, the machine learning model 1630 may generate the oocyte quality information 1632 that includes at least an oocyte grade 1680, which can indicate a likelihood of the oocyte developing into a usable blastocyst. As illustrated, the oocyte grade 1680 may be one of Grade A, Grade B, Grade C, or Inconclusive (INC) that will be explained in more detail below. The oocyte grade 1680 can be presented to a user via an interactive user interface for further analysis.

As illustrated in FIG. 16B, the segmentation model 1640 may process the image sequences 1602 (e.g., including the image 1604A through the image 1604N) of the oocyte 1610 to identify the objects 1660 associated with the oocyte 1610 (e.g., objects that represent different parts of the oocyte). More specifically, the objects 1660 identified by the segmentation model 1640 may include a bounding box (BBOX) 1660A, a zona pellucida (ZP) 1660E, a perivitelline space (PVS) 1660D, a first polar body (FPB) 1660B, and a cytoplasm (CPM) 1660C. In some examples, the segmentation model 1640 may utilize machine learning algorithms or architectures, such as U-Net architecture optionally with a MobileNet_v2 encoder backbone, to generate segmentation masks for identifying the BBOX 1660A, FPB 1660B, CPM 1660C, PVS 1660D, and ZP 1660E. As may be appreciated, the segmentation masks may help in isolating and identifying different parts of the oocyte.

In some examples, the segmentation model 1640 may be trained, tuned, and/or validated using machine learning techniques. Training the segmentation model 1640 may include data selection, model training, and model validation to ensure that the segmentation model 1640 can accurately segment and identify the BBOX 1660A, FPB 1660B, CPM 1660C, PVS 1660D, and ZP 1660E. Data selection may include collecting and organizing a dataset of oocyte images that can be used for model training and model validation. The dataset may include images from various sources (e.g., humans, cow, pig oocytes) to ensure diversity and robustness. The images may be annotated with bounding boxes, first polar body, cytoplasm, perivitelline space, zona pellucida. Raw images and annotated images may further be separated for better data control and management using Git and Data Version Control (DVC). The dataset may be split into training sets and validation sets.

Once the dataset is prepared, the segmentation model 1640 may be trained. The model training process may involve selecting an appropriate model architecture (e.g., a convolutional neural network such as a U-Net, a vision transformer architecture, or the like), defining hyperparameters (e.g., encoder depth, decoder channel, batch size, initial learning rate, optimizer, scheduler, or the like), and using data augmentation techniques (e.g., Grid distortion, optical distortion, random crop, shift scale rotate) to improve model performance. Additionally, a combination of loss functions, such as Multiclass Focal Loss and Dice Loss, can be used to handle class imbalance and improve segmentation accuracy. The training progress may be monitored using metrics like mean Intersection over Union (mIOU) and F1-Score.

After training the segmentation model 1640, the segmentation model 1640 can be validated using the validation dataset. The validation process may ensure that the segmentation model 1640 generalizes well to unseen data and can accurately segment different parts of oocytes. Based on the data selection and management, model training, and model validation processes described above, the segmentation model 1640 can accurately identify and segment various parts of the oocyte, enabling precise feature extraction and grading. This, in turn, aids in the objective determination of oocyte grades, improving the chances of successful in-vitro fertilization (IVF) treatments.

In some examples, the ZP 1660E may be the outer layer of the oocyte and may protect the oocyte and/or facilitate sperm binding during fertilization. The PVS 1660D may be the space between the ZP 1660E and the CPM 1660C. The PVS 1660D may contain the FPB 1660B. The FPB 1660B may be a relatively small cell that is extruded from the oocyte during meiosis. The presence and morphology of the FPB 1660B can provide insights into the oocyte's developmental potential. The CPM 1660C may contain various organelles. The CPM 1660C may be critical for the oocyte's metabolic activities and developmental competence.

Based on some or all of the BBOX 1660A, FPB 1660B, CPM 1660C, PVS 1660D, and ZP 1660E identified by the segmentation model 1640, the feature extractor 1650 may determine the features 1622 associated with the oocyte 1610. More specifically, the features 1622 may be determined or calculated based on various combinations of measurements and/or geometry information associated with the objects 1660 (e.g., the BBOX 1660A, FPB 1660B, CPM 1660C, PVS 1660D, and ZP 1660E). The features 1622 may include morphological features indicative of measurements of the oocyte 1610 and an aspiration depth (D) of the oocyte 1610 shown in FIG. 14. In some examples, the morphological features may be associated with the objects 1660 identified by the segmentation model 1640. For example, the morphological features may include at least one of an ellipticity of the FPB 1660B, a thickness of the ZP 1660E, a diameter of the oocyte 1610, an area of the CPM 1660C, a compactness of the CPM 1660C, a circularity of the CPM 1660C, and a ratio between the area of the CPM 1660C and a total area of the CPM 1660C and the PVS 1660D.

Based on the features 1622, the machine learning model 1630 may generate the oocyte quality information 1632 that includes at least the oocyte grade 1680. In some examples, the machine learning model 1630 may be a regression model. The regression model may include a plurality of weights. In some examples, the plurality of weights may be iteratively adjusted through training processes associated with the regression model. Each of the plurality of weights may be associated with one of the features 1622 for determining the oocyte grade 1680. For example, a first weight may be used to multiply a first feature (e.g., the ellipticity of the FPB 1660B) to generate a first product, a second weight may be used to multiply a second feature (e.g., the area of the CPM 1660C) to generate a second product, and so forth. The oocyte grade 1680 may be derived by summing the first product, the second product, and so forth.

In some examples, as noted above, the oocyte grade 1680 may categorize the quality of oocytes into a threshold number of grades (e.g., 2, 3, 4, 10 grades, and so on): A, B, C, and Inconclusive (INC). The Grades A, B, and C may represent the likelihood of an oocyte developing into a usable blastocyst, which is useful for successful in-vitro fertilization (IVF) treatments. In some embodiments, the grade 1680 may represent a value which is assigned into a particular range reflecting one of the four grades or a different number of grades.

Grade A may represent the highest likelihood of developing into a usable blastocyst. An oocyte with Grade A may exhibit optimal morphological and mechanical features, such as ideal ellipticity of the first polar body, appropriate thickness of the zona pellucida, and/or favorable compactness and circularity of the cytoplasm. The high-quality metrics associated with Grade A oocytes suggest a strong potential for successful fertilization and subsequent embryo development.

An oocyte with Grade B may have a good likelihood of developing into a usable blastocyst, though not as high as Grade A oocytes. Grade B oocytes may still exhibit favorable morphological and mechanical features, but may have minor deviations from the optimal values seen in Grade A oocytes. Despite these minor deviations, Grade B oocytes may still be considered viable and have a reasonable chance of successful fertilization and embryo development.

Oocytes classified as Grade C may have a lower likelihood of developing into a usable blastocyst. Grade C oocytes may exhibit several deviations from the optimal morphological and mechanical features, such as irregular ellipticity of the first polar body, suboptimal thickness of the zona pellucida, and less favorable compactness and circularity of the cytoplasm. While Grade C oocytes may not be ideal, they may still have some potential for successful fertilization and embryo development, albeit with a lower probability compared to Grade A and Grade B oocytes.

Oocytes classified as Inconclusive (INC) may have an uncertain likelihood of developing into a usable blastocyst. This classification may result from insufficient or ambiguous data (e.g., blurred image sequence), making it challenging to accurately assess the oocyte's quality. Inconclusive oocytes may require further analysis or additional data to determine their viability. The INC grade indicates that the current assessment does not provide a definitive conclusion about the oocyte's potential for successful fertilization and embryo development. By categorizing oocytes into these grades, the oocyte analysis system 1600 provides a more objective, automated, and time-efficient evaluation of oocyte quality, aiding embryologists and clinicians in making informed decisions during the IVF process.

### Example Flowcharts

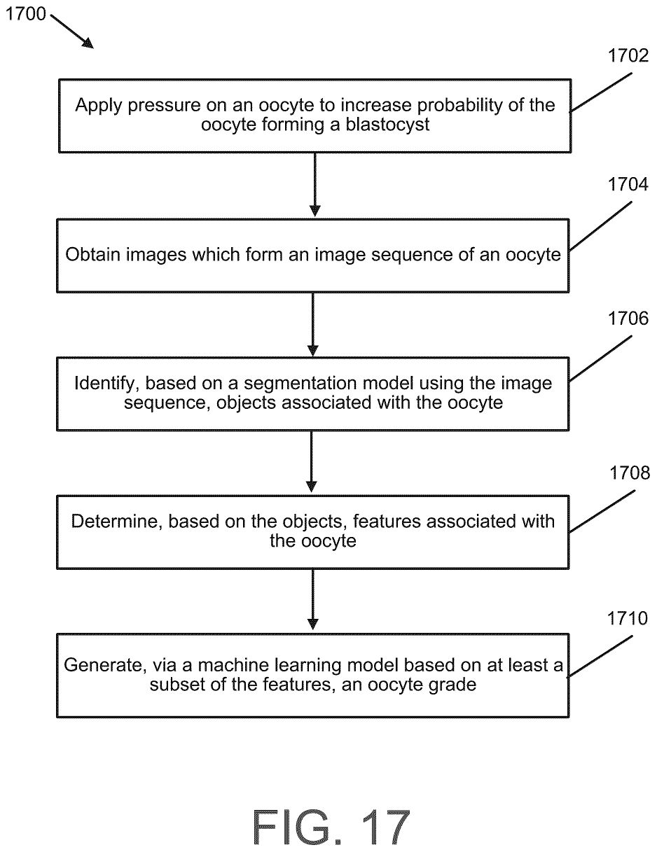

FIG. 17 is a flowchart of an example process 1700 for applying pressure on an oocyte to increase a probability of the oocyte forming a blastocyst and generating an oocyte grade for the oocyte. All or at least some parts of the process 1700 may be implemented, for example, by a pressure generating device (e.g., the pressure generating device 1) and oocyte analysis system 1600 of FIGS. 16A and 16B (collectively referred to as a “system”). Advantageously, the process 1700 may boost chances of the oocyte (e.g., the oocyte 1610) forming a blastocyst and/or provide for determining viability (e.g., whether an oocyte will develop into a useful blastocyst) of the oocyte without resorting to manual and subjective assessment by embryologists.

At block 1702, the system applies pressure on an oocyte to increase a probability of the oocyte forming a blastocyst. For example, the pressure generating device may apply a slight negative pressure that is generated according to Table 1 to the oocyte 1610 through the micropipette 3, thereby increasing the probability of the oocyte 1610 forming a blastocyst. In some embodiments, the process 1700 may end after block 1702.

In some examples, the oocyte analysis system 1600 can determine, or otherwise select, a particular pressure profile, pressure range, and/or time range (e.g., selecting among pressure profile, ranges, and time ranges shown in Table 1) based on the oocyte quality information 1632 for boosting blastocyst formation of the oocyte 1610. For example, based on the oocyte grade 1680 generated by the machine learning model 1630 associated with corresponding pressures applied by the pressure generating device to oocytes (e.g., block 1710), the oocyte analysis system 1600 can determine that applying a pressure between a pressure range from a first value to a second value, or a constant value, s more likely to lead to better oocyte grade 1680 (e.g., Grade A), compared with applying other pressure ranges to an oocyte. In this example, the oocyte analysis system 1600 can control the pressure generating device (e.g., the pressure generating device 1) to apply a pressure with pressure values between the first value and the second value, or the constant value, to the oocyte 1610 through a micropipette for boosting blastocyst formation.

As another example, the oocyte analysis system 1600 can select or determine a pressure range and/or time range based on experiment results. In this example, experiment results may show that applying a pressure with pressure values between a first value (e.g., −0.2 psi) and a second value (e.g., −0.1 psi) to an oocyte can result in 56% of the oocyte forming a blastocyst, and applying a pressure with pressure values between other values (e.g., −0.3 psi to −0.2 psi) to an oocyte can result in 45% of the oocyte forming a blastocyst. Based on the experiment results, the oocyte analysis system 1600 can control the pressure generating device to apply a pressure with pressure values between −0.2 psi to −0.1 psi to the oocyte 1610 through a micropipette for boosting blastocyst formation. In this way, the oocyte analysis system 1600 may determine pressure values, ranges, and so on; time values, ranges, and so on.

At block 1704, the system obtains images which form an image sequence of an oocyte. As discussed above, the images (e.g., the image sequence 1602) may be captured by microscopic cameras and depict a sequence of events showing geometry information, deformation, and/or movements of an oocyte resulted from application of forces on the oocyte by a tool (e.g., the micropipette 3). In some examples, the system obtains a plurality of images which form an image sequence associated with a time period. This image sequence depicts an oocyte (e.g., the oocyte 1610) and a portion of a tool (e.g., the micropipette 3) applying pressure to the oocyte. Each individual image in the sequence may be associated with individual pressure values applied to the oocyte at the respective times of image capture. It will be understood that block 1702 and block 1704 can be performed concurrently or at least partially overlapping in time. For example, while applying the pressure (e.g., negative pressure) on the oocyte, images forming an image sequence of the oocyte can be obtained simultaneously.

At block 1706, the system identifies, based on a segmentation model using the image sequence of the oocyte, objects associated with the oocyte. In some examples, the system identifies objects associated with the oocyte via the segmentation model 1640. These objects can include various parts of the oocyte such as the zona pellucida (e.g., ZP 1660E), perivitelline space (e.g., PVS 1660D), first polar body (e.g., FPB 1660B), cytoplasm (e.g., CPM 1660C), and a bounding box (e.g., BBOX 1660A) associated with the portion of the micropipette applying the pressure to the oocyte.

At block 1708, the system determines features (e.g., the features 1622) associated with the oocyte based on the objects identified. In some examples, the features 1622 can include morphological features indicative of measurements of the oocyte during the time period and an aspiration depth of the oocyte into the portion of the micropipette 3 applying the pressure. The morphological features can include features such as the ellipticity of the first polar body, thickness of the zona pellucida, diameter of the oocyte, area of the cytoplasm, compactness of the cytoplasm, circularity of the cytoplasm, and a ratio between the area of the cytoplasm and the total area of the cytoplasm and the perivitelline space.

At block 1710, the system generates an oocyte grade via a machine learning model based on at least a subset of the features determined at block 1708. In some examples, the system generates an oocyte grade (e.g., the oocyte grade 1680) via the machine learning model 1630 based on input comprising at least a subset of the features 1622. In some embodiments, the subset may include only the aspiration depth. In some embodiments, the subset may include the aspiration depth and at least one other feature. The oocyte grade 1680 may be indicative of at least a likelihood of the oocyte developing into a usable blastocyst. The machine learning model 1630 can be a regression model that includes a plurality of weights, each associated with one of the features, to generate the oocyte grade 1680. The oocyte grade 1680 can then be provided via an interactive user interface for further analysis.

### Example System Block Diagram

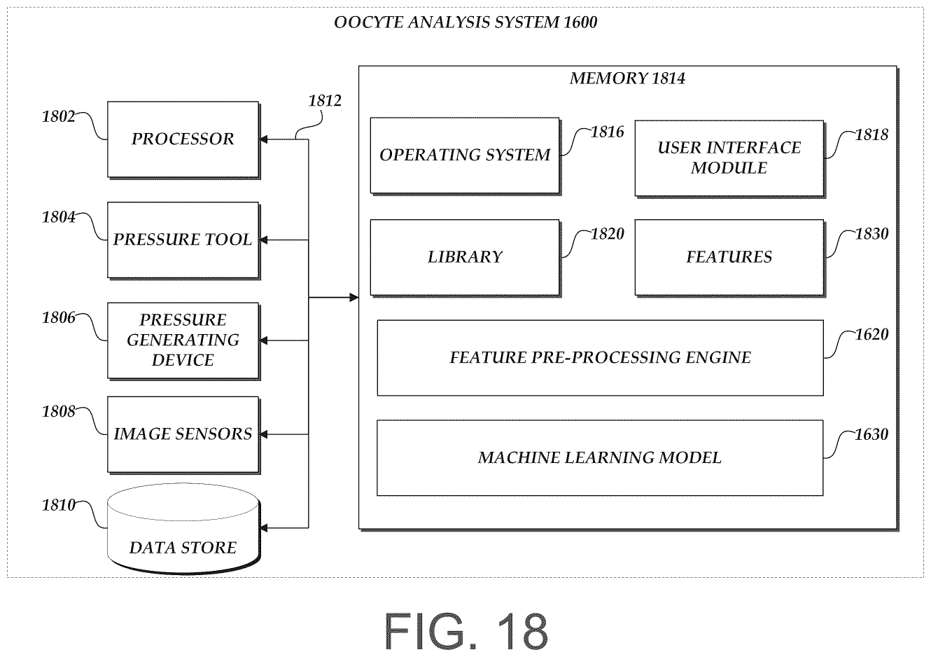

FIG. 18 depicts a general architecture of an example system. The system may be used, in some embodiments to perform the functionality described herein. In some embodiments, the system may be the oocyte analysis system 1600, which includes an arrangement of computer hardware and software such as a pressure generating device and a micropipette configured to implement aspects of the present disclosure. The oocyte analysis system 1600 may include more (or fewer) elements than those shown in FIG. 18. It is not necessary, however, that all of these elements be shown in order to provide an enabling disclosure.

As illustrated, the oocyte analysis system 1600 includes a processor 1802, a pressure tool 1804 (e.g., the micropipette 3 of FIGS. 13, 14 and 16A), a pressure generating device 1806 (e.g., the pressure generating device 1 of FIG. 1 or any other pressure generating device), image sensors 1808 (e.g., one or more microscope cameras used to capture the image sequence 1602 of FIG. 16A) and a data store 1810, all of which may communicate with one another by way of a communication bus 1812. The pressure tool 1804 may not be included in some embodiments and the oocyte analysis system 1600 may represent a back-end processing system. As noted above, the pressure generating device 1806 may be the pressure generating device 1 of FIG. 1 or any other pressure generating device that can generate pressure (e.g., pressure generated according to Table 1) to be applied (e.g., through the pressure tool 1804 such as the micropipette 3) on an oocyte for increasing a probability of the oocyte forming a blastocyst. In some embodiments, the oocyte analysis system 1600 may be configured to process requests from other devices or modules through a network interface that is not shown in FGI. 18, such as requests to analyze oocyte qualities from a remote device or server. The data store 1810 may illustratively be any non-transitory computer-readable data store, and in various embodiments may store any or all of the elements that are depicted in FIG. 18 as being loaded into a memory 1814.

The processor 1802 may also communicate to and from the memory 1814. The memory 1814 may contain computer program instructions (grouped as modules or components in some embodiments) that the processor 1802 may execute in order to implement one or more embodiments. The memory 1814 generally includes RAM, ROM, and/or other persistent, auxiliary, or non-transitory computer-readable media. The memory 1814 may store an operating system 1816 that provides computer program instructions for use by the processor 1802 in the general administration and operation of the oocyte analysis system 1600. The memory 1814 may further store specific computer-executable instructions and other information (which may be referred to herein as “modules” or “engines”) for implementing aspects of the present disclosure. For example, the memory 1814 may include the feature pre-processing engine 1620 and the machine learning model 1630, which may implement aspects of the present disclosure as described above. The memory 1814 may further store, for example, user interface module 1818 that may enable presentation of information to a user interface of a user device (not shown in FIG. 18). In addition, the memory 1814 can store the library 1820 (e.g., for storing parameters of different types of machine learning models) and features 1830 (e.g., including at least the features 1622 of FIG. 16B) that may be extracted by the feature pre-processing engine 1620. All of the modules or elements loaded into the memory 1814 may also be stored in the data store 1810 as various operations are performed.

It will be recognized that many of the components described in FIG. 18 are optional and that embodiments of the oocyte analysis system 1600 may or may not combine components. Furthermore, components need not be distinct or discrete. Components may also be reorganized. In some embodiments, components illustrated as part of the oocyte analysis system 1600 may additionally or alternatively be included in other computing devices, such that some aspects of the present disclosure may be performed by the oocyte analysis system 1600 while other aspects are performed by another computing device.

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or media or device (e.g., solid state storage devices, disk drives, etc.). The various functions disclosed herein may be embodied in such program instructions, or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

The processes described herein or illustrated in the figures of the present disclosure may begin in response to an event, such as on a predetermined or dynamically determined schedule, on demand when initiated by a user or system administrator, or in response to some other event. When such processes are initiated, a set of executable program instructions stored on one or more non-transitory computer-readable media (e.g., hard drive, flash memory, removable media, etc.) may be loaded into memory (e.g., RAM) of a server or other computing device. The executable instructions may then be executed by a hardware-based computer processor of the computing device. In some embodiments, such processes or portions thereof may be implemented on multiple computing devices and/or multiple processors, serially or in parallel.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware (e.g., ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements or steps. Thus, such conditional language is not generally intended to imply that features, elements or steps are in any way required for one or more embodiments or that one or more embodiments necessarily include logic for deciding, with or without other input or prompting, whether these features, elements or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, or Z). Thus, such disjunctive language is not generally intended to, and should not, imply that certain embodiments require at least one of X, at least one of Y, and at least one of Z to each be present.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. All changes which come within the meaning and range of equivalency of the claims are to be embraced within their scope.

### Example Clauses

Examples of implementations of the present disclosure can be described in view of the following example clauses. The features recited in the below example implementations can be combined with additional features disclosed herein. Furthermore, additional inventive combinations of features are disclosed herein, which are not specifically recited in the below example implementations, and which do not include the same features as the specific implementations below. For sake of brevity, the below example implementations do not identify every inventive aspect of this disclosure. The below example implementations are not intended to identify key features or essential features of any subject matter described herein. Any of the example clauses below, or any features of the example clauses, can be combined with any one or more other example clauses, or features of the example clauses or other features of the present disclosure.

Clause 1. A pressure generating device, comprising:
a tank having a chamber therein, and including an opening and a communication port each of which is in fluid communication with said chamber;
a deformable membrane which is disposed to seal said opening, and which is deformable between a flat state and a deformed state;
a driving device which is mounted to said tank and which includes a motor body and a driving shaft, said driving shaft having a first shaft end that is disposed to confront said deformable membrane, said driving shaft being driven by said motor body to move between a distal position, where said first shaft end is distal from said motor body, and a proximate position, where said first shaft end is proximate to said motor body; and
a connection unit configured to couple said second shaft end with said deformable membrane and to permit said deformable membrane to be driven by said driving shaft to transform between the flat state, where said first shaft end is in one of the distal portion and the proximate portion, and the deformed state, where said first shaft end is in the other one of the distal position and the proximate position, such that a predetermined pressure is generated through said communication port when said deformable membrane is moved from one of the flat state and the deformed state to the other one of the flat state and the deformed state.

Clause 2. The pressure generating device as claimed in Clause 1, wherein said deformable membrane is deformed inwardly in the deformed state.

Clause 3. The pressure generating device as claimed in Clause 1, wherein said deformable membrane is deformed outwardly in the deformed state.

Clause 4. The pressure generating device as claimed in Clause 1, wherein:
said tank includes
   a main casing having said chamber therein, and including said opening and said communication port, and
   a side casing detachably mounted to said main casing such that said deformable membrane is secured between said main casing and said side casing to thereby seal said opening;
said motor body is immovably mounted to said side casing; and
said driving shaft extends along a shaft axis, and is driven by said motor body to move toward or away from said deformable membrane so as to transform said deformable membrane between the flat state and the deformed state.

Clause 5. The pressure generating device as claimed in Clause 4, wherein said main casing extends along a longitudinal axis to terminate at a closed end and a connection end formed with said opening.

Clause 6. The pressure generating device as claimed in Clause 4, wherein:
said side casing includes
   a side casing body that has a distal end and a proximal end opposite to said distal end, and that is formed with a passage extending from said distal end to said proximal end, and
   a flange body that extends radially and outwardly from said proximate end of said side casing body, and that is detachably mounted to said main casing; and
said motor body has an inner portion received in said passage of said side casing body,
an outer portion extending from said inner portion in a direction away from said main casing, and disposed outside said side casing body, and
a flange portion extending radially from said outer portion, and detachably secured to said distal end of said side casing body.

Clause 7. The pressure generating device as claimed in Clause 1, wherein said connection unit includes:
a connection sleeve which is coupled on said first shaft end of said driving shaft so as to permit said connection sleeve to move with said driving shaft, said connection sleeve having a first sleeve portion, a second sleeve portion having an outer dimension smaller than an outer dimension of said first sleeve portion, and a shoulder surface between said first sleeve portion and said second sleeve portion; a bearing sleeve which is fittingly sleeved on said second sleeve portion so as to permit said bearing sleeve to move with said connection sleeve, and which is disposed between said connection sleeve and said deformable membrane, said bearing sleeve having a first abutment end that is in abutting engagement with said shoulder surface, and a second abutment end that is opposite to said first abutment end; and
a bearing cap having an engaging portion formed with a recess that is configured to engage with said second abutment end so as to permit said bearing cap to move with said bearing sleeve, and
a mounted portion which is opposite to said engaging portion, and which is secured to a first surface of said deformable membrane so as to transmit a force from said driving shaft to said deformable membrane.

Clause 8. The pressure generating device as claimed in Clause 7, wherein said deformable membrane is formed with a through hole, and said connection unit further includes a fastener having
an enlarged head which is disposed on a second surface of said deformable membrane opposite to said first surface, and
a fastening rod extending from said enlarged head through said through hole of said deformable membrane to terminate at a rod end, said rod end being fittingly engaged within a hole formed in said mounted portion of said bearing cap so as to permit said deformable membrane to be fastened to said bearing cap through said fastener.

Clause 9. The pressure generating device as claimed in Clause 7, wherein said connection unit further includes
a first washer disposed between said protruded portion and said first surface of said deformable membrane, and
a second washer disposed between said enlarged head and said second surface of said deformable membrane.

Clause 10. The pressure generating device as claimed in Clause 1, further comprising a photo sensor which is immovable relative to said tank so as to permit said photo sensor to detect a displacement of said driving shaft.

Clause 11. The pressure generating device as claimed in Clause 10, further comprising a flag piece which is mounted on a second shaft end of said driving shaft opposite to said first shaft end so as to permit said flag piece to move with said driving shaft, and which has a plurality of detectable positions to be detected by said photo sensor, thereby determining the displacement of said driving shaft.

Clause 12. The pressure generating device as claimed in Clause 1, wherein
a hardness of said deformable membrane ranges from 0 Shore A to 100 Shore A,
a thrust of said driving device ranges from 100 g to 1000 g, and
a stroke of said driving device ranges from 3 mm to 50 mm.

Clause 13. A detecting system for detecting quality of a test sample including an oocyte or an embryo, comprising:
said pressure generating device as claimed in Clause 1; a micropipette for sucking the test sample; and
a connecting tube disposed to connect said communication port of said tank to said micropipette for applying the predetermined pressure to the test sample.

Clause 14. The detecting system as claimed in Clause 13, wherein an inner diameter of said micropipette ranges from 10 microns to 100 microns.

Clause 15. The detecting system as claimed in Clause 13, wherein a pressure ranging from 0.5 psi to −0.5 psi is generated at a suction port of the micropipette.

Clause 16. The detecting system as claimed in Clause 15, wherein the connecting tube includes a first tube segment connected to the communication port of the tank, and a second tube segment connected to the micropipette, said first and second tube segments being connected to each other through an adjusting valve for adjusting the pressure generated at said suction port of the micropipette.

What is claimed is:

1. A method implemented by a system of one or more computers, the method comprising:
applying, through a micropipette, pressure that is generated by a pressure generating device to an oocyte;
obtaining a plurality of images which form an image sequence associated with a time period, the image sequence depicting the oocyte and a portion of the micropipette applying the pressure to the oocyte, wherein individual images are associated with individual pressure values applied to the oocyte at respective times of image capture;
identifying, via a segmentation model, objects associated with the oocyte;
determining, based at least in part on geometry measurements of the objects associated with the oocyte, features associated with the oocyte, the features comprising morphological features indicative of measurements of the oocyte during the time period and an aspiration depth of the oocyte into the portion of the micropipette applying the pressure to the oocyte; and
generating, via a machine learning model based on input comprising the aspiration depth, an oocyte grade, wherein the oocyte grade is indicative of at least a likelihood of the oocyte developing into a usable blastocyst.

2. The method of claim 1, wherein the pressure is between −0.5 psi to 0.5 psi.

3. The method of claim 1, wherein the pressure is applied on the oocyte for 0.5 seconds to 10 seconds.

4. The method of claim 1, wherein the pressure is generated by the pressure generating device according to a two-step pressure profile or a continued pressure profile.

5. The method of claim 1, wherein an inner of the micropipette is between 25 microns to 100 microns.

6. The method of claim 1, wherein the objects associated with the oocyte include at least one of a zona pellucida of the oocyte, perivitelline space of the oocyte, a first polar body of the oocyte, a cytoplasm of the oocyte, and a bounding box associated with the portion of the micropipette applying the pressure to the oocyte.

7. The method of claim 6, wherein the morphological features associated with the oocyte include at least one of an ellipticity of the first polar body, a thickness of the zona pellucida, a diameter of the oocyte, an area of the cytoplasm, a compactness of the cytoplasm, a circularity of the cytoplasm, and a ratio between the area of the cytoplasm and a total area of the cytoplasm and the perivitelline space.

8. The method of claim 1, wherein the segmentation model comprises a U-net, and the machine learning model comprises a regression model.

9. The method of claim 1, wherein applying the pressure to the oocyte increases a probability of the oocyte forming a blastocyst.

10. A method implemented by a system of one or more computers for increasing a probability of an oocyte forming a blastocyst, the method comprising:
applying, through a micropipette, pressure that is generated by a pressure generating device to an oocyte,
wherein applying the pressure to the oocyte increases the probability of the oocyte forming the blastocyst.

11. The method of claim 10, wherein the pressure generating device comprises:
a tank having a chamber therein, and including an opening and a communication port each of which is in fluid communication with said chamber;
a deformable membrane which is disposed to seal said opening, and which is deformable between a flat state and a deformed state;
a driving device which is mounted to said tank and which includes a motor body and a driving shaft, said driving shaft having a first shaft end that is disposed to confront said deformable membrane, said driving shaft being driven by said motor body to move between a distal position, where said first shaft end is distal from said motor body, and a proximate position, where said first shaft end is proximate to said motor body; and
a connection unit configured to couple said first shaft end with said deformable membrane and to permit said deformable membrane to be driven by said driving shaft to transform between the flat state, where said first shaft end is in one of a distal portion and a proximate portion, and the deformed state, where said first shaft end is in the other one of the distal position and the proximate position, such that a predetermined pressure is generated through said communication port when said deformable membrane is moved from one of the flat state and the deformed state to the other one of the flat state and the deformed state.

12. The method of claim 10, further comprising:
obtaining a plurality of images which form an image sequence associated with a time period, the image sequence depicting the oocyte and a portion of the micropipette applying the pressure to the oocyte, wherein individual images are associated with individual pressure values applied to the oocyte at respective times of image capture;
identifying, via a segmentation model, objects associated with the oocyte;
determining, based at least in part on geometry measurements of the objects associated with the oocyte, features associated with the oocyte, the features comprising morphological features indicative of measurements of the oocyte during the time period and an aspiration depth of the oocyte into the portion of the micropipette applying the pressure to the oocyte; and
generating, via a machine learning model based on input comprising the aspiration depth, an oocyte grade, wherein the oocyte grade is indicative of at least a likelihood of the oocyte developing into a usable blastocyst.

13. The method of claim 10, wherein the pressure is between −0.5 psi to 0.5 psi.

14. The method of claim 10, wherein the pressure is applied on the oocyte for 0.5 seconds to 10 seconds.

15. The method of claim 10, wherein the pressure is generated by the pressure generating device according to a two-step pressure profile or a continued pressure profile.

16. The method of claim 10, wherein an inner of the micropipette is between 25 microns to 100 microns.

17. A system comprising one or more processors and non-transitory computer storage media storing instructions that when executed by the one or more processors, cause the one or more processors to:
apply, through a micropipette, pressure that is generated by a pressure generating device to an oocyte,
wherein applying the pressure to the oocyte increases a probability of the oocyte forming a blastocyst.

18. The system of claim 17, wherein the instructions further cause the one or more processors to:
obtain a plurality of images which form an image sequence associated with a time period, the image sequence depicting the oocyte and a portion of the micropipette applying the pressure to the oocyte, wherein individual images are associated with individual pressure values applied to the oocyte at respective times of image capture;
identify, via a segmentation model, objects associated with the oocyte;
determine, based at least in part on geometry measurements of the objects associated with the oocyte, features associated with the oocyte, the features comprising morphological features indicative of measurements of the oocyte during the time period and an aspiration depth of the oocyte into the portion of the micropipette applying the pressure to the oocyte; and
generate, via a machine learning model based on input comprising the aspiration depth, an oocyte grade, wherein the oocyte grade is indicative of at least a likelihood of the oocyte developing into a usable blastocyst.

19. The system of claim 17, wherein the pressure generating device comprises:
a tank having a chamber therein, and including an opening and a communication port each of which is in fluid communication with said chamber;
a deformable membrane which is disposed to seal said opening, and which is deformable between a flat state and a deformed state;
a driving device which is mounted to said tank and which includes a motor body and a driving shaft, said driving shaft having a first shaft end that is disposed to confront said deformable membrane, said driving shaft being driven by said motor body to move between a distal position, where said first shaft end is distal from said motor body, and a proximate position, where said first shaft end is proximate to said motor body; and
a connection unit configured to couple said first shaft end with said deformable membrane and to permit said deformable membrane to be driven by said driving shaft to transform between the flat state, where said first shaft end is in one of a distal portion and a proximate portion, and the deformed state, where said first shaft end is in the other one of the distal position and the proximate position, such that a predetermined pressure is generated through said communication port when said deformable membrane is moved from one of the flat state and the deformed state to the other one of the flat state and the deformed state.

20. The system of claim 17, wherein the pressure is applied to the oocyte for 0.5 seconds to 10 seconds.
